(19) United States
(12) Patent Application Publication
(10) Pub. No.: US 2010/0305389 A1
(43) Pub. Date: Dec. 2, 2010

(54) Device Suitable for Introducing into Human and/or Animal Bodies

(76) Inventors: Hans-Dieter Voss, Rosenheim (DE); Juergen Albert, Karlsruhe (DE); Jakob Jilinski, Pforzheim (DE)

Correspondence Address: BOHAN MATHERS, PO BOX 17707, PORTLAND, ME, 04112-8707, US

(21) Appl. No.: 12/664,144

(22) PCT Filed: Jun. 11, 2008
(86) PCT No.: PCT/DE08/00949

§ 371 Date: May 27, 2010

(30) Foreign Application Priority Data
Jun. 11, 2007 (DE) 10 2007 027 285.7

(51) Int. Cl. A61N 5/00

(52) U.S. Cl. 600/3

(57) ABSTRACT

The invention relates to a device suitable for at least partially introducing into human and/or animal bodies, said device containing barium iodide located at least on one wall such that the device is visible during an imaging method.

### BACKGROUND INFORMATION

1. Field of the Invention

The present invention relates to a device that is suitable for insertion at least partially into a human or animal body. More particularly, the invention relates to a method of using barium iodide together with the device, so as to render the device or a hollow space within the body more readily visible with conventional imaging technologies.

2. Description of the Prior Art

Devices that are suitable for insertion at least partially into the human or animal body are well-known in the prior art. Such devices include, for example, angioplasty balloons, stents, pacemakers, artificial joints, catheters, etc. Such devices are often made of materials such as plastic, titanium, nitinol, etc., and, because of that, are only poorly visible in imaging procedures, such as, CT scans or X-rays.

### BRIEF SUMMARY OF THE INVENTION

It is the goal of the invention to provide a device that is suitable for insertion at least partially into a human or animal body, one that is better recognizable by means of imaging procedures. This goal is achieved by providing a device for insertion into the body and which contains some form of barium iodide.

The device according to the invention contains barium iodide, or barium iodide is provided on at least one wall of the device. The barium iodide may be provided as a surfacial or a dotted application in the device or on the wall of the device. The barium iodide may also lie loosely in and/or on the device.

The term barium iodide as used hereinafter shall generally refer to the chemical substance, as well as to barium iodatum.

Barium iodide is readily visible in imaging procedures, such as, X-ray, CT scan, etc., and also has an antiseptic effect. Providing barium iodide on at least one wall of the device makes that wall readily visible with the aforementioned procedures. Even when the device contains barium iodide, the parts that contain barium iodide are easily recognizable in such imaging procedures. This facilitates the control of the device, both when introducing into, as well as positioning the device in the human or animal body.

Barium iodide has antiseptic properties, so if it intentionally or unintentionally gets into the human or animal body, it prevents infections from developing in the body.

If the barium iodide is provided on the walls, the device can first be manufactured by means of the extrusion process, etc., and the barium iodide subsequently applied. This may be advantageous, because the barium iodide is applied in a second step and is thus not subject to the manufacturing constraints for making the device.

Overall, the barium iodide may be present in its three physical aggregation states, thus, as a solid, a liquid, and a gas, but also as a liquid crystal.

Preferably, the device has a hollow space defined by a wall or walls, whereby barium iodide is provided on the wall or walls and/or in the hollow space. Although the barium iodide may be provided on the inner, as well as the outer, walls of the hollow space, it is preferable that the barium iodide be provided on the inner walls of the hollow space. The outer wall of the device will thus prevent the barium iodide from coming into contact with the human or animal body.

In a preferred embodiment, the hollow space of the device is expandable and contractable. The hollow space may be expanded either by adding a liquid and/or expand by itself due to the mechanical properties of the hollow space. Thus, the hollow space may be inserted into the human or animal body in a contracted form and be expanded subsequently, for example, by adding a liquid, and thereby assume a functional form. The hollow space may also expand by prestressing it during insertion, and then, inside the body, removing a prestressing mechanism that prevents expansion, so as to allow the hollow space to then expand inside the body.

In another special embodiment, the barium iodide is present in powder form. In this powder form, the barium iodide is also visible in imaging procedures, but only weak signals during the imaging procedure are the result, when the powder covers only a small portion of the device. Nevertheless, the barium iodide as powder may be provided on the device in such a way that it takes up the smallest possible space during insertion of the device and thus does not needlessly enlarge the device. If an imaging procedure is used for monitoring purposes, a liquid may be added to the barium iodide. This facilitates an even distribution of the barium iodide throughout a larger space, which can thus be seen more clearly during the imaging procedures, for example, X-rays and/or CT scans, etc. A common NaCl solution may be used for this purpose, but the liquid may also include solvents, such as alcohol.

The barium iodide, particularly when it is in powder form, may also be distributed by means of a gas, so that it is distributed over a larger area and contributes to stronger signals in the imaging procedures, such as CT scans and X-rays.

Generally, it is also one aspect of the invention to provide, in a preliminary step, a radiographic contrast medium on the device that is suitable for insertion into the human or animal body and, subsequently, if so desired, to render this device visible by means of an imaging procedure. The radiographic contrast medium may be, for example, a powder. This preliminary step may be made visible by adding a liquid, for example, to the radiographic contrast medium.

In another preferred embodiment, the barium iodide is in a liquid-soluble form. This enables the barium iodide to be dissolved and distributed within the hollow space by a liquid that is added, when, for example, a hollow space is to be expanded like a balloon.

The barium iodide may also be present at least partially in dissolved form. Thus, the barium iodide may be present, already dissolved, for example, in a higher concentration than is necessary for the expanded form of the hollow body. This can prevent the formation of clumps, for example, when a liquid is added, and ensure that the barium iodide is evenly distributed within the hollow body.

The barium iodide may also be dissolved such that the hollow space is visible in the CT scan and/or the X-ray, even when it is not yet expanded. In addition, the barium iodide may be further thinned by adding more liquid and/or gas, and thereby continue to make the now expanded, larger hollow space visible.

A hollow space that is suitable for attachment to the device that is suitable for insertion at least partially into the human or animal body may also be contain barium iodide. The barium iodide may be brought into the device by means of a liquid that flows through the hollow space, thus making the device visible in imaging procedures. Such a hollow space may be a multi-path flow control device, such as a tap or cock, for example, also with a bypass, but also another hollow space, such as a one-way tap, a chamber, a valve, a branch line, whereby the barium iodide is provided in the multi-path tap or in parts of the multi-path tap or in the hollow space or in the bypass. The barium iodide may be in a powder form, in another solid form, or also in the form of a liquid or a gas.

The hollow space may also be a syringe, whereby here, too, the barium iodide may be in the form of a powder, as well as in another solid form, but also in the form of a liquid, liquid crystals, and/or a gas. The syringe may also already be provided with a barium iodide solution, so that the barium iodide may be injected with the solution into the aforementioned device by means of the syringe.

The barium iodide may be provided either in a highly concentrated form or in a ready-to-use solution. The ready-to-use solution is particularly suitable for interaction with electromagnetic radiation, especially X-rays. The ready-to-use solution should particularly exhibit the property of high visibility with imaging procedures that use ionizing or electromagnetic radiation.

It is also an aspect of the invention to provide barium iodide dissolved in a liquid for use as the radiographic contrast medium. It is also an aspect of the invention to provide barium iodide dissolved in a liquid for use as an expanding liquid for expandable hollow bodies that are suitable for insertion into the human or animal body. The solution liquid may particularly contain NaCl. The solution liquid may also contain water, an alcohol, such as, methanol, ethanol, propanol, etc., and/or a solvent.

Furthermore, it is another aspect of the invention to provide barium iodide for producing a radiographic contrast medium. The barium iodide may be provided in a solid form, for example, as wire, and/or in powder form, and/or as a liquid, and/or as liquid crystal, and/or as gas.

Another aspect of the invention is to provide barium iodide for producing an expanding liquid for the purpose of expanding an at least partially expandable hollow space that is suitable for insertion into the human or animal body. Here, too, the barium iodide may be entirely or partially in solid form, for example, as wire, in liquid form, in liquid-crystal form, and/or in the form of a gas.

Another aspect of the invention is to provide a carrier body and to use the carrier body for storing, transporting, and/or metering the barium iodide. The carrier body may be a body made of stainless steel, fused quartz, titanium, or similar inert and/or stable materials. The carrier body may also be dust-like or comprise numerous small carrier bodies and/or have numerous cavities, particularly be sponge-like. Due to the special embodiments of the carrier body, the carrier body has a large surface to which the barium iodide may be attached.

The carrier body may be used as follows. In a first step, the barium iodide is dissolved and/or dispersed in a liquid. Such a liquid can, for example, be water and/or alcohol. The carrier bodies are moistened with the liquid, so that the liquid attaches to the surface of the carrier bodies or penetrates into the corresponding cavities. The barium iodide is separated from the liquid in a drying process, for example, by means of air-drying and/or vacuum-drying, and remains on the surface of the carrier body. The carrier body, or a majority of the carrier bodies, treated in this manner may be introduced into a cavity. The amount of barium iodide thereby stored by the carrier bodies may be determined by the number or quantity of carrier bodies. By means of this cavity, which may also possibly be provided with a screen device, a liquid, such as, water, alcohol, etc., is fed into the device for introduction into the human or animal body. The barium iodide is thereby absorbed by the liquid and transported further into the aforementioned device. Preferably, the cavity is arranged external to the body. This enables easy storage and metering of the barium iodide. The use of carrier bodies is generally not limited to barium iodide. The inventive idea may also be realized with numerous liquid-soluble substances.

### BRIEF DESCRIPTION OF THE DRAWINGS

The invention is explained in more detail below based on multiple drawings, whereby the drawings do not represent limitations to the scope of protection.

FIG. 1A is an illustration of a balloon catheter in its contracted form, with barium iodide introduced into the balloon.

FIG. 1B is an illustration of the balloon catheter of FIG. 1A, in its expanded form.

FIG. 2A is an illustration of a balloon catheter in its contracted form, with a three-way tap.

FIG. 2B is an illustration of the balloon catheter of FIG. 2A, in its expanded form.

FIG. 3A is an illustration of a balloon catheter in its contracted form, connectible to a syringe that contains barium iodide.

FIG. 3B is an illustration of the devices in FIG. 3A, with the syringe also filled with a solution.

FIG. 3C is an illustration of the devices of FIG. 3B, with the balloon catheter in its expanded form.

### DETAILED DESCRIPTION OF THE PREFERRED EMBODIMENTS

Before the invention is described in detail, it is noted here that the components and the procedural steps of the invention may vary and it is not intended that the scope of protection be limited to the respective components of the device or to the respective procedural steps described hereinafter. The terms used here are merely intended to describe special embodiments and are not used as limitations. Furthermore, the use of a singular or indefinite article in the description or the claims shall also encompass the plural of the elements, as long as the overall context does not clearly indicate something different.

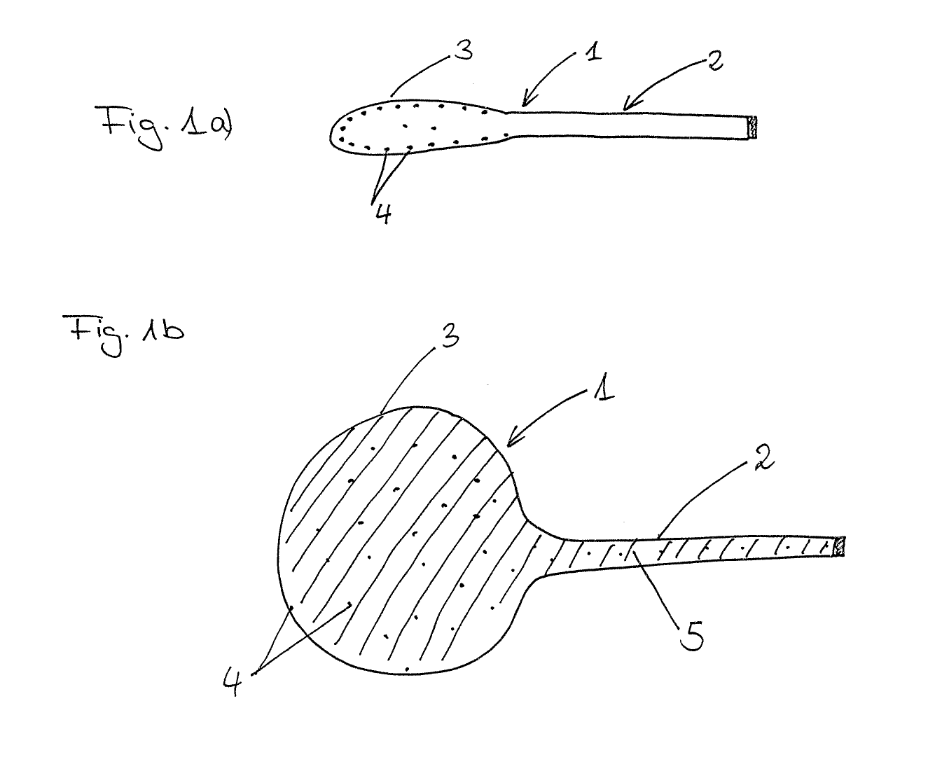

FIG. 1A shows a hollow insertion device 1, in this case, a balloon catheter 1, in a contracted, or non-expanded form, whereby the balloon catheter 1 comprises a catheter 2 and an expandable and contractable balloon 3. Barium iodide 4 in powder form has been applied to the wall of the balloon 3. The barium iodide 4 is therefore found, at least partially, on the balloon wall.

FIG. 1B also shows the balloon catheter 1 of FIG. 1A, in its expanded state, whereby liquid 5, for example, a NaCl solution, has been introduced into the catheter 2. The barium iodide 4 dissolves in the liquid 5 and is evenly distributable throughout the balloon 3, thus rendering the balloon 3 quite visible in imaging procedures. It is also possible that the barium iodide 4 not be introduced directly into the balloon 3, but rather be introduced into the catheter 2, and carried into the balloon 3 by the solution 5, where it is distributed evenly throughout the balloon 3.

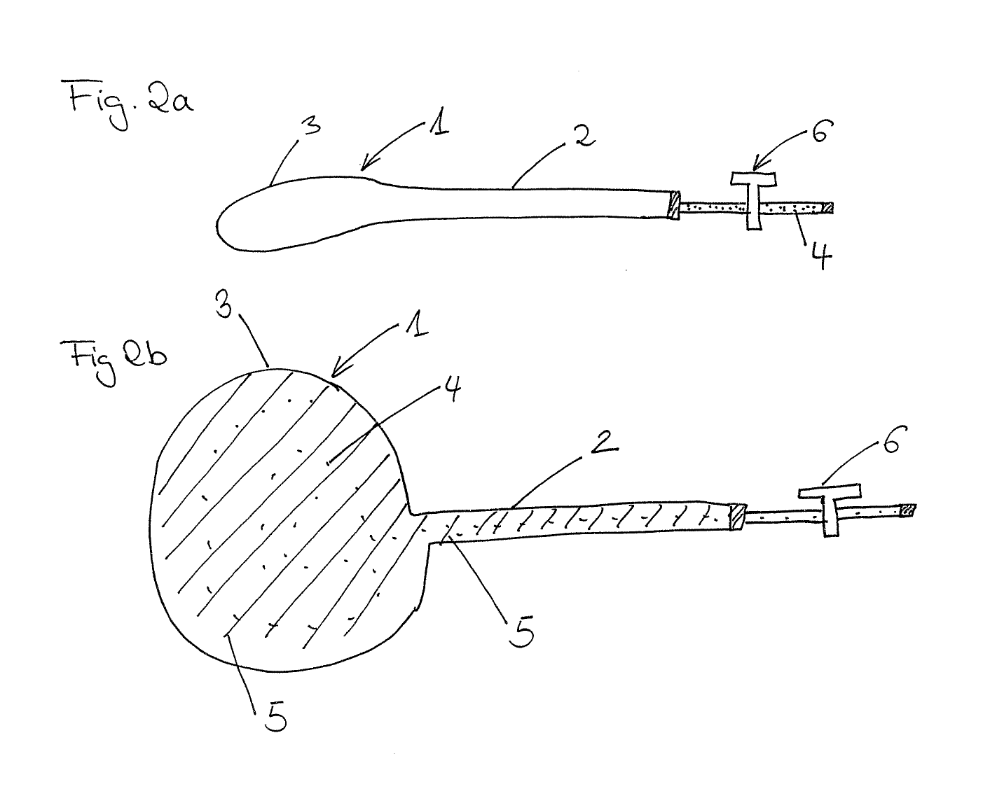

FIGS. 2A and 2B show the balloon catheter of FIGS. 1A and 1B, used together with a peripheral device 6, in which barium iodide 4 is introduced in pure or dissolved form. In the embodiments shown in FIGS. 2A and 2B, the peripheral device 6 is a flow control device, such as a multi-path cock or tap 6, that is peripheral to the insertion device 1 and that has a capacity for storing a liquid substance. Instead of the multi-path tap, other flow control devices 6, for example, a one-way tap, a valve, or a branch line, may also be used to hold the barium iodide 4 for a certain period of time.

In particular, the peripheral device 6 may be made available in a pre-packaged and/or sterilized form, whereby the amount of barium iodide 4 present in the peripheral device 6 is adjusted appropriately to accommodate the capacity of the corresponding balloon 3 or the respective device 1 that is to be made visible in the imaging procedure. For different insertion devices 1 that are to be made visible, it is thereby also possible to provide different and/or the same peripheral devices 6 with different amounts of barium iodide 4 that have been adjusted for the respective insertion devices 1.

The association of the peripheral device or flow control device 6 to the corresponding insertion device 1 that is to be made visible during the imaging procedure may be indicated on the packaging for the peripheral device 6, on the peripheral device 6 itself, etc. The peripheral device 6 may also be constructed in such a way that it may be connectible only to a corresponding insertion device 1, such as a specific balloon catheter 1.

Introducing the liquid or gas into the balloon 3 by means of the flow control device 6 also carries the barium iodide 4 into the balloon 3, whereby the barium iodide 4, along with the liquid 5, is distributed evenly throughout the inner volume of the balloon 3, thus making the balloon 3 visible in the imaging procedure.

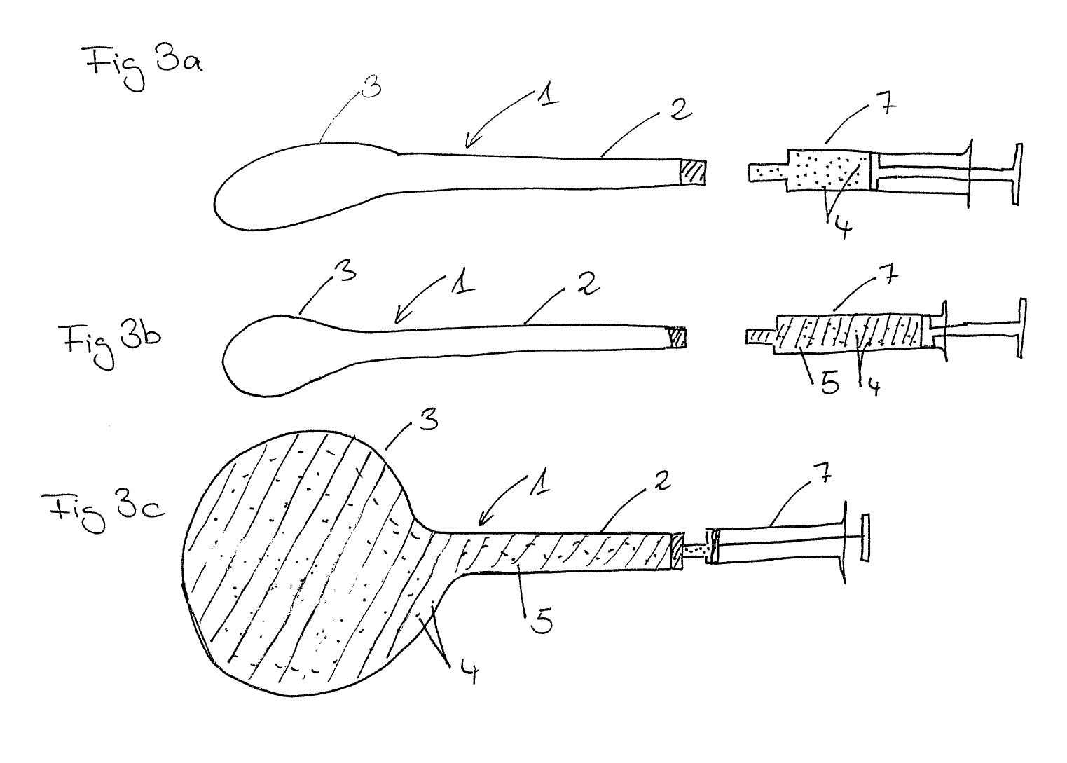

FIGS. 3A-3C show the insertion device/balloon catheter 1 and the peripheral device/flow control device 6. In the embodiment shown, the peripheral device 6 is a syringe 7 that contains barium iodide 4. The barium iodide 4 may either be filled into the syringe 7 shortly before introducing the catheter 2 into the human or animal body, or the syringe 7 may be supplied pre-filled with a predetermined amount of barium iodide.

In particular, the syringe 7 may be provided as a package pre-filled with the barium iodide and/or sterilized. Here, too, barium iodide 4 may again be used in powder form and/or in another solid form and/or as liquid crystals and/or in dissolved form or as a gas.

Now, if a liquid 5 is added to the syringe 7, then the barium iodide 4 binds with the liquid 5, or rather, is dissolved in this liquid 5. The liquid 5 may either be provided in a separate container that is connected to the syringe 7, or the container may be attached to a connector piece, for example, a flow control device 6, i.e., a multi-path tap 6 between the syringe 7 and the balloon catheter 1, so that the liquid 5 flows into the syringe 7 when the syringe 7 is pulled apart. The syringe 7 may be manufactured in such a way that the required amount of liquid 5 is determined by the size of the syringe 7.

FIG. 3B shows the syringe 7, which now contains barium iodide 4 diluted by means of the liquid 5. This liquid 5 can now be introduced into the balloon catheter 1 by means of the syringe 7 and thereby render the balloon catheter 1 visible in the CT scan or the X-ray. Filling the liquid 5 into the balloon catheter 1 expands the balloon 3 and, in the case of a vascular balloon, thereby stretches the vascular wall.

Generally, it is also possible that the syringe 7 or the flow control device 6 be distributed for sale as a pre-manufactured device including the barium iodide 4. In order to make the balloon 3 visible, the syringe 7 is either filled with a predetermined amount of liquid 5, then attached directly to the balloon catheter 1, or attached via one or more connector means or connector pieces and then the liquid 5 with the barium iodide 4 introduced into the balloon catheter 1.

The connector piece may also be provided with barium iodide 4 and then supplied as a pre-filled barium-iodide piece. In this case, a predetermined amount of liquid 5 is conducted through the connector piece, so that the barium iodide 4 binds with the liquid 5 and flows into the balloon catheter 1.

1. Device (1) suitable for insertion at least partially into the human and/or animal body, characterized in that the device contains barium iodide (4) or that barium iodide (4) is provided on at least one wall.

2. Device according to claim 1, characterized in that the device has a hollow space (3) and that barium iodide is provided on the walls of the hollow space (3).

3. Device according to the preceding claims, characterized in that the device (1) has an expandable and contractable hollow space (3), such as, for example, a vascular balloon.

4. Device according to one of the preceding claims, characterized in that the barium iodide (4) is in powder form.

5. Device according to one of the preceding claims, characterized in that the barium iodide (4) is soluble in a liquid (5).

6. Device according to one of the preceding claims, characterized in that the barium iodide (4) is at least partially in dissolved form.

7. Hollow space (6,7) that is suitable for being connected to a device (1) according to the preceding claims, characterized in that barium iodide (4) is present in this hollow space (6,7).

8. Hollow space (6,7) according to the preceding claim, characterized in that the hollow space (6) is a multi-path tap (6).

9. Hollow space (6,7) according to one of the preceding claims 7 and 8, characterized in that the hollow space (7) is a syringe.

10. Hollow space (6,7) according to one of the preceding claims 7 to 9, that the barium iodide (4) present in the hollow space (6,7) is at least partially in powder form.

11. Hollow space (6,7) according to one of the preceding claims 7 to 10, characterized in that barium iodide (4) is at least partially in dissolved form.

12. Hollow space (6,7) according to the preceding claim, characterized in that barium iodide is a ready-to-use solution.

13. Hollow space (6,7) according to the preceding claim, characterized in that the ready-to-use solution interacts with electromagnetic radiation.

14. Hollow space (6,7) according to the preceding claim, characterized in that the ready-to-use solution interacts with X-ray radiation.

15. Barium iodide (4) for use as an X-ray contrast medium.

16. Barium iodide (4) dissolved in a liquid (5), for use as an X-ray contrast medium.

17. Barium iodide (4) dissolved in a liquid (5), for use as an expanding liquid for expandable hollow bodies (3) that are suitable for being inserted into a human and/or animal body.

18. Solution according to claim 16 or 17, characterized in that the liquid (5) contains NaCl.

19. Solution according to claims 16 to 18, characterized in that the liquid (5) contains H₂O.

20. Solution according to claims 16 to 19, characterized in that the liquid (5) contains a solvent such as alcohol.

21. Barium iodide in powder form for producing an X-ray contrast medium.

22. Barium iodide in powder form for producing an expanding fluid for the expansion of an at least partially expandable hollow body that is suitable for being inserted into a human and/or animal body.

23. Carrier body for dissolvable binding of barium iodide, characterized in that the carrier body has numerous cavities, particularly is sponge-like.

24. Carrier body for dissolvable bonding of barium iodide, characterized in that the carrier body is essentially dust-like, particularly with particle sizes smaller than 1 mm, preferably smaller than 0.1 mm.

25. Carrier body according to claim 23 or 24, characterized in that the body is essentially made of fused quartz and/or titanium and/or stainless steel.
